(19) United States
(12) Patent Application Publication
(10) Pub. No.: US 2026/0026722 A1
(43) Pub. Date: Jan. 29, 2026

(54) ELECTROLYTE FOR AN ELECTROCHEMICAL GAS SENSOR AND BLOOD GAS MONITORING

(71) Applicant: Sentec AG, Therwil (CH)

(72) Inventors: Amirhossein DROUDIAN, Thalwil (CH); Nadezda PANKRATOVA, Witterswil (CH); Victoria Emily DE LANGE, Olten (CH); Patrick BOSSHARD, Olten (CH); Peter Matthias SCHUMACHER, Kirchlindach (CH)

(73) Assignee: Sentec AG, Therwil (CH)

(21) Appl. No.: 18/994,263

(22) PCT Filed: Jul. 15, 2022
(86) PCT No.: PCT/EP2022/069891

§ 371 Date: Jan. 14, 2025

(51) Int. Cl. A61B 5/1477; A61B 5/1455; C08K 3/16; C08K 3/26; C08K 5/053; C08L 71/02; G01N 27/404

(52) U.S. Cl. CPC A61B 5/1477; A61B 5/14552; C08K 3/16; C08K 3/26; C08K 5/053; C08L 71/02; G01N 27/4045; C08K 2003/262; C08K 2201/014; C08L 2201/54; C08L 2203/20

(57) ABSTRACT

An electrolyte composition for use in an electrochemical sensor, said electrolyte composition comprising at least one hygroscopic compound comprising at least two moieties (HB) with hydrogen bonding capability and at least one hydrophilic evaporation-inhibiting compound comprising a carbon-to-HB ratio of ≥2 and a molecular weight MW of more than 100 g/mol, wherein said evaporation-inhibiting compound comprises a lower viscosity and a lower surface tension than said hygroscopic compound.

The present invention relates to an electrolyte for an electrochemical gas sensor, an electrochemical gas sensor for blood gas monitoring, a method of sensing a target gas partial pressure, and the use of an electrolyte according to the preamble of the independent claims.

Electrochemical sensors allow the measurement of an analyte by means of two or more electrodes connected by an electrolyte, whereby at least one of the electrodes represents the indicator electrode, at which the analyte to be determined alters chemically and creates a potential shift on the electrode. Electrochemical sensors have proven to be particularly suitable for measuring gaseous analytes.

Gases, particularly CO₂ and O₂ play an important role on physiology of different functions of living bodies. Therefore, technologies such as electrochemical-based gas sensing found their way in medical device sensors, enabling monitoring of these respiratory gases, among them sensors for measuring blood gases transcutaneously, at which the sensor measures carbon dioxide and oxygen diffused out from the blood to the skin surface.

In these medical devices, an electrolyte defines the interface between gases and sensing electrodes. Therefore, electrolyte media need to fulfil certain criteria in order to enable stable measurements over relatively long application times as well as provide adequate response time at the general clinical conditions. The primary criteria include the stability of the electrolyte composition, adequate solubility of the respective target gas in the electrolyte, temperature stability, chemical inertness, adequate ionic conductivity, suitable surface tension, and non-toxicity of the electrolyte components, where unintentional patient or living body contact can bring significant risks.

For blood gas monitoring, the measurement of carbon dioxide or its partial pressure (pCO₂) can be carried out by using potentiometric sensors comprising at least one indicator electrode and at least one reference electrode. For Stow-Severinghaus-type electrodes, the measurement of the CO₂ partial pressure is based on a pH measurement. This generally requires a reaction chamber, which is spatially separated from the analysing medium by a gas-permeable and largely ion-impermeable membrane. In this reaction chamber the pH value is measured in an electrolyte with a mild buffer solution containing sodium bicarbonate. Carbon dioxide diffuses through the membrane and into the electrolyte where it partially converts into carbonic acid following to reactions:

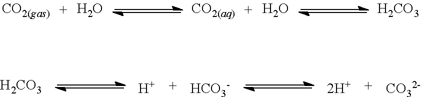

At a given temperature, initial pH and composition of the electrolyte in the reaction chamber, the pH value of the reaction chamber depends on the CO₂ partial pressure of the sample.

Water, a key part of an electrolyte's composition, participates on one hand in the CO₂ hydrolysis reaction, and on the other hand influences significantly the activity of ions and proton (H⁺). Therefore, maintaining a constant water content in the electrolyte is a key factor in reducing measurement drift over time enabling reliable and fast sensing. This is particularly important in the field of medical sensors used for gas sensing as these sensors conventionally need to undergo frequent calibration with dry gas with known composition, which makes it even more difficult to maintain a constant water content in the electrolyte.

To minimize the evaporation of water, hygroscopic electrolytes have been used, which attract and hold water molecules via eithe absorption or adsorption from the surrounding environment. Furthermore, gas permeable membranes have been used to cover the electrolyte and further reduce evaporation.

Such liquid electrolyte compositions for electrochemical gas sensors, where the electrolytes comprise a hygroscopic matrix of ethylene glycol and/or propylene glycol and an aqueous salt solution to increase ionic conductivity, are known from EP 3 151 000 A1, for example.

However, the electrolytes known from the state of the art suffer from certain drawbacks, which consequently also extend to the electrochemical sensors and the clinical measurements in which they are used. For instance, electrolytes based on high vapour pressure compounds such as ethylene glycol or propylene glycol evaporate within a few days of measurement. The evaporation leads to a couple of known drawbacks. First, it changes the electrolyte's relative composition and therefore ionic activities, which leads to a drift and makes frequent calibration of the sensor necessary. Second, with advanced evaporation, previously dissolved salts precipitate from the electrolyte leading to deposits on the electrodes and structural surfaces of the sensor. This phenomena not only makes the sensors responding slower over time, but also the precipitated salts dissolve again after reapplication of a new electrolyte leading to an additional composition change with related drift and inaccuracy issues. Overall, related issues originating from electrolyte evaporation dictate frequent sensor calibration and maintenance. This maintenance translates into a tedious workload for clinicians in the stressful patient monitoring conditions, hindering usability of the medical device. The state of the art products require weekly to monthly exchange of electrolyte, beyond which the sensor operates unreliably.

To address the evaporation issue in the general electrochemical sensor research field, electrolytes based on hydrogels and solid-state compounds have been proposed (M. Tierney et al., Anal. Chem 1993, 65, 23, 3435-3440; U. Guth et al., J. Solid State Electrochem. 2009, 13, 27-39). However, the reported hydrogels suffer from high response time over long time application, which is either due to the low ability of hydrogels to retain water over a longer measurement period of several days and/or the high viscosity of hydrogels effecting low ionic conductivity. In contrast, solid-state electrolytes have no problem with evaporation but operate at temperatures typically above 80° C. Although these advancements avoid electrolyte evaporation, their performances, i.e. response time, or their operating conditions such as 80° C. working temperature prevent their implementation in transcutaneous blood gas monitoring, where the main application of this invention focuses on. In transcutaneous monitoring, the sensor stays in contact with skin and needs to be fast enough to reflect physiological changes.

From the processing and manufacturing point view, the electrolyte formulation should enable facile application of the electrolyte on the sensor. Here, two main important properties namely viscosity and surface tension play a major role. Viscosity affects the rheology of electrolyte. Generally, moderate viscosity is required to apply a thin layer and to keep the electrolyte on the sensor, without letting it falling away from the reaction chamber during application. On the other hand, the formulation needs to have moderate surface tension to cover different parts of the sensor with variable hydrophobicity and hydrophilicity. Bubbles can form in case of a mismatch between sensor surface properties and the electrolyte coating capability. Such bubbles lead to inaccurate measurement and drift over time due to their gas composition mismatch and partitioning with the electrolyte as well as their movement in the electrolyte over time.

Overall, clinical application and manufacturability of electrochemical sensors dictate certain molecular properties to the electrolyte as the heart of the electrochemical sensors. The properties ideally include but are not limited to:
1. low vapour pressure
2. high density of hydrogen bonds
3. high ionic mobility and sensitivity
4. elevated hygroscopicity
5. moderate viscosity
6. tuneable surface tension
7. chemical stability at the clinical condition i.e. oxidation/decomposition resistance, compatibility with sensor components
8. Low toxicity

It is thus an object of the present invention to remedy these and other disadvantages of the state of the art and in particular to provide an electrolyte composition and an electrochemical gas sensor with improved clinical usability, which allow for a reliable and stable measurement of target gases, fast clinically acceptable response times, which in the context of respiratory gas monitoring typically means a few seconds to minutes, and extended intervals between electrolyte changes, maintenance and calibrations. Further objects of the present invention are to provide a method for sensing gases, and to propose the use of an electrolyte, which is safe and easy to use for the detection of at least one target gas, in particular carbon dioxide and/or oxygen.

The object is achieved by an electrolyte composition, an electrochemical gas sensor for CO₂ detection, optionally in combination with an optical sensor for O₂ measurement, a method of sensing a target gas, and the use of an electrolyte pursuant to the independent claims. Advantageous embodiments are subject of the dependent claims.

According to the present invention, the electrolyte composition for use in an electrochemical sensor comprises at least one hygroscopic compound with at least two moieties (HB) having hydrogen bonding capability and at least one hydrophilic evaporation-inhibiting compound with a carbon-to-HB ratio of 22 and a molecular weight MW of more than 100 g/mol. The evaporation-inhibiting compound comprises a lower viscosity and a lower surface tension than said hygroscopic compound.

It has now been surprisingly found that such an electrolyte composition features enough gas solubility to enable gas sensing, negligible vapour pressure to keep the composition of the electrolyte constant over a prolonged measuring period and thus minimize the drift of the measurement, and is hygroscopic enough to retain water over several days of measuring and upon calibration of the electrochemical sensor with both wet and dry gases. Such electrolyte fulfils the requirements for clinical evaluation of blood gases based on electrochemical gas sensing.

The electrolyte composition according to the invention is particularly suitable for use in sensors for the transcutaneous measurement of blood gases, as these sensors are exposed to frequent changes between wet and dry environments. A wet environment is caused or provided, for example, by patient sweat, application sealing liquid or gel, and humid monitoring condition such as incubators, while the dry environment is present, for example, during calibration, storage or transfer of the sensor.

Preferably, the at least one hygroscopic compound of the electrolyte composition disclosed herein features a carbon-to-HB ratio of ≤2, more preferably ≤1.5, even more preferably ≤1.34, and most preferably 1.0.

By using compounds with these features, the hygroscopicity of the electrolyte composition can be further increased, allowing moisture to be retained even longer. In addition, a higher number of HB moieties, i.e. a low carbon-to-HB ratio, allows even faster proton transport and thus even faster measurements of gases whose electrochemical measurement principle is based on the transport of protons and hydrogen bonding, as in the case of the measurement of CO₂. Hydrogen bonds contribute to proton mobility and therefore have a positive effect on the response time of the electrochemical sensor, as will be described in more detail further below.

Preferably, the at least one hydrophilic evaporation-inhibiting compound of the electrolyte composition disclosed herein with a carbon-to-HB ratio of 22 has a molecular weight of more than 400 g/mol.

A higher molecular weight results in a lower volatility of the compound and thus contributes to the stability of the electrolyte composition.

Optionally, the hygroscopic compound of the electrolyte composition disclosed herein has a proton diffusion coefficient of more than 10⁻⁷ cm²/s, preferably of more than 10⁻⁶ cm²/s, at 25° C.

A higher proton mobility has a positive effect on the response time of the electrochemical sensor, as will be described in more detail further below.

In the context of the present specification, the moieties (HB) having hydrogen bonding capability may be hydrogen bond donors (HBDs) and/or hydrogen bond acceptors (HBAs). Preferably, the moieties having hydrogen bonding capability are at least hydrogen bond acceptors (HBAs). The moieties having hydrogen bonding capability are part of the compounds that make up the electrolyte, i.e. they are covalently connected to the rest of the respective molecules. The respective compounds can have different, i.e. mixed, or identical moieties having hydrogen bonding capability. Each moiety is capable of forming at least one hydrogen bond with a moiety of the same or a different type or water. Hydrogen bonding may be intermolecular or intramolecular, albeit the positive effects of the hydrogen bonds on the electrolyte properties described herein are particularly due to intermolecular hydrogen bonding.

There are two general mechanisms responsible for proton mobility in proton-conducting electrolytes: the vehicle mechanism (which relies on the physical transport of a vehicle to move protons) and the Grotthuss mechanism, which involves the proton being handed-off from one hydrogen bonding site to another (A. Hassanali et al., PNAS 2013,110 (34), 13723-13728). The Grotthuss mechanism depends on a site-to-site hopping mechanism of protons (H⁺), while the vehicle mechanism depends on the rate of physical diffusion of the vehicle i.e. H₃O⁺. Vehicles are simply molecules that are capable of forming a bond to the free proton and of freely diffusing. Previous research demonstrated that protons hop over hydrogen bonds and therefore hydrogen bonds enhance proton mobility based on the Grotthuss mechanism (Ghosh et al. Chem. Mater 2005, 17 (3), 661-669, M. McDonnell et al., J. Phys. Chem. B 2016, 120, 5223-5242).

In a preferred embodiment of the electrolyte disclosed herein, said moieties having hydrogen bonding capability are at least one of hydroxyl, carboxyl, amine, imine, and/or amide groups. It is particularly advantageous if the moieties having hydrogen bonding capability are selected from hydroxyl, carboxyl, or amine moieties.

Moreover, said hygroscopic compound is preferably selected from the group consisting of polyhydric alcohols, glycerol, triglycerol, polyglycerol, diethylene glycol, triethylene glycol, triethanolamine, diethanolamine and/or propylene glycol. More preferably, said hygroscopic compound is selected from polyhydric alcohols, glycerol, triglycerol, diethylene glycol, triethylene glycol and/or diethanolamine. Most preferably, said hygroscopic compound is glycerol and/or diethylene glycol. Additionally or alternatively, the hygroscopic compound is preferably present in the electrolyte in an amount of less than 90% based on the total weight of the electrolyte.

The use of ultra-hydrophilic and hygroscopic compounds such as glycerol and diethylene glycol, which both feature a carbon-to-HB ratio of equal or smaller than 2 and concomitantly low vapour pressures, allows for prolonged retention of humidity in the electrolyte composition. In addition, the high hydrogen-bond density enable a fast transport of protons and therefore fast sensing of gases such as CO₂. Furthermore, these compounds are non-toxic and largely chemically inert.

In a preferred embodiment of the electrolyte disclosed herein, the evaporation-inhibiting compound is selected from the group consisting of short chain (oligomers) of polyethylene glycol, polyglycerol, polyoxazolines, polyhydroxy-functional acrylates, in particular poly(2-hydroxyethyl methacrylate), polyethylene oxide, hydrophilic polycarbonates and co-, graft- and/or block copolymers thereof.

Hydrophilic polycarbonates comprising hydroxyl groups in polymer side chains may be prepared as described by Engler et al. (Macromolecules 2015, 48 (6), 1673-1678).

The use of these evaporation-inhibiting compounds has the advantage that these compounds have a lower viscosity, surface tension and hydrophilicity in comparison to strongly hygroscopic compounds such as glycerol. As a consequence, the creation of bubble-free layers on sensor structures featuring relative hydrophobicity is greatly facilitated. Especially the use of polyethylene glycol (PEG) is preferred according to the invention, as its end-groups are capable of forming hydrogen bonds and can thus contribute to proton transport yet their main chains are amphiphilic and soluble in water as well as in many organic solvents. Furthermore, even short chain PEGs are viscous liquids that have a low vapour pressure. For sensing oxygen importantly PEGs have a higher O₂ solubility compared to strongly hygroscopic compounds such as glycerol. Hence, the use of PEGs allows for clinically acceptable response times in the sensing of O₂, when optical-based oxygen sensing is embedded in the electrolyte.

Preferably, the evaporation-inhibiting compound is present in the electrolyte in an amount of at least 10% based on the total weight of the electrolyte.

At this concentration in the electrolyte, the advantageous properties of the evaporation-inhibiting compound become particularly apparent.

In a preferred embodiment, the electrolyte further comprises a surfactant.

By using a surfactant in the electrolyte composition, the surface tension of the electrolyte composition can be adjusted. This can improve the coatability of ultra-hydrophobic surfaces such as membranes made from polytetrafluoroethylene (PTFE) with hydrophilic electrolyte compositions. For example, the use of surfactants may assist in the creation of coatings without air inclusions in the coating, which is of great importance for the measuring accuracy of unknown gas composition using the electrochemical sensor, as will be described in more detail later.

Preferably, the surfactant in the electrolyte composition disclosed herein is a non-ionic and non-metallic surfactant. This has the advantage that undesirable interactions with the measuring principle are avoided.

Different compounds are suitable to be considered as the surfactant either alone or as a mixture. In a preferred embodiment, the surfactant used in the electrolyte composition disclosed herein is selected from the group consisting of long-chain (macromolecules) of polyvinylpyrrolidone (PVP), polyvinyl alcohol (PVA), polysaccharides, and co-, graft- and/or block copolymers thereof. It is particularly advantageous if the surfactant is selected from the group consisting of polyvinylpyrrolidone (PVP), polyvinyl alcohol (PVA), polysaccharides and co-, graft- and/or block copolymers thereof.

These surfactants have the advantage that they do not negatively influence the measuring principle and that they are commercially readily available.

The surfactant is preferably present in the electrolyte in an amount of less than 5%, more preferably in an amount of less than 2%, based on the total weight of the electrolyte. In these preferred concentration ranges an optimal effectiveness of the surfactants used was found.

Another property of the electrolyte that is to be taken into account for the coatability of substrates, such as the surfaces of sensors, is the viscosity of the electrolyte composition. For example, the viscosity of the electrolyte influences the achievable final thickness of the electrolyte coating after application and therefore sensor response time, as electrolyte thickness defines the time required for ions and gases to diffuse through and equilibrate. Electrolyte thickness might also be defined with a porous spacer material expanding over pH sensor and contains the electrolyte. In this case, viscosity affects intrusion of the electrolyte in the pores of spacer and uniform wetting of the holes.

In a preferred embodiment, the electrolyte composition disclosed herein further comprises a thickening agent.

By using a thickening agent in the electrolyte composition, the viscosity of the electrolyte can be tuned by changing the concentration or molecular weight of the respective thickening agent. Thus, leaking of the electrolyte out of the sensor or through the membrane can be prevented, for example.

Preferably, the thickening agent in the electrolyte composition disclosed herein is a non-ionic and non-metallic thickening agent.

Non-ionic and non-metallic thickening agents have the advantage that they do not negatively influence the measuring principle and that they are commercially readily available.

It is of course also conceivable and according to the invention that more than one surfactant and/or thickening agent is used in the electrolyte composition disclosed herein.

In a preferred embodiment, the thickening agent is selected from the group consisting of polyvinylpyrrolidone (PVP), polyethylene derivatives, polyvinyl alcohols, poly-(2-vinylpyridines), polyethylene oxides, polyethylene glycol monomethyl ethers, polyvinyl methyl ethers, polysaccharides, polycarbonates, and combinations thereof.

Preferably, the thickening agent in the electrolyte composition disclosed herein is selected from the group consisting of polyvinylpyrrolidone (PVP), polyethylene derivatives, polysaccharides and combinations thereof.

Examples of polysaccharides preferred by the invention include cellulose, starch, dextranes, cellulose derivatives, starch derivatives, and dextroses.

The thickening agent is preferably present in the electrolyte in an amount of less than 5%, more preferably in an amount of less than 2%, based on the total weight of the electrolyte.

In a preferred embodiment, the electrolyte composition disclosed herein further comprises an aqueous solution of at least one inorganic bicarbonate-based pH buffer, in particular with a buffer range between pH 7 and 9.

Such pH buffer systems, which can be used for liquid electrolyte solutions, include any bicarbonate-based buffer system known in the field, which serves to semi-stabilize the pH value in an aqueous medium.

Depending on the electrode materials used in the electrochemical sensor, it may be advantageous to add chlorides to the aqueous buffer solution, for example when using Ag/AgCl as (pseudo) reference electrode, in direct contact with the electrolyte.

Optionally, the aqueous solution further comprises at least one chloride salt, wherein the concentration of said chloride salt in the aqueous buffer solution is less than 3 mol/L.

Chlorides serve as conducting salt component of the liquid electrolyte, providing stability of the silver chloride electrode and enhancing ionic strength, which is beneficial for faster system response time.

Preferably the concentration of chloride salt in the aqueous buffer solution is less than 1 mol/L.

In a preferred embodiment of the electrolyte composition disclosed herein, the bicarbonate-based pH buffer is selected from the group consisting of sodium bicarbonate buffer and/or potassium bicarbonate buffer. Alternatively or in addition, the chloride salt is selected from the group consisting of LiCl, NaCl, and/or KCl.

These buffer systems have the advantage that they buffer the pH value of the electrolyte in the preferred range from pH 7 to 9, when CO₂ is added to system, enabling stable detection of this gas. The preferred chlorides have the advantage that they are water-soluble and available at very low cost. Especially NaCl and/or KCl are preferred alkali metal chlorides, as they offer the advantage to provide stable potential for the reference electrode.

In a preferred embodiment, the evaporation-inhibiting compound of the electrolyte composition disclosed herein has an oxygen solubility of more than 0.01 mg/L and less than 10 mg/L at 25° C. and 1 bar, i.e. atmospheric pressure.

Such an electrolyte composition is particularly well suited for the sensing of oxygen.

In addition or alternatively, all compounds in the electrolyte composition disclosed herein—with the exception of water—have a vapor pressure smaller than 0.1 mmHg at 25° C.

The use of compounds, which fulfil this property are particularly suitable for use in the electrolyte according to the invention as they allow for particularly stable sensing measurements with small drift.

In the context of the present specification, the values given for the vapor pressure refer to values obtained by manometric measurement.

Other suitable auxiliaries that can be added to the electrolyte composition include defoamers. By adding small quantities of at least one defoaming agent, the wetting of surfaces with the electrolyte can be improved.

In a preferred embodiment, the electrolyte comprises less than 90% of said hygroscopic compound, preferably glycerol, more than 10% of said hydrophilic evaporation-inhibiting compound, preferably polyethylene glycol, less than 10% of an aqueous solution of NaHCO₃ and KCl, and less than 5% of said surfactant, preferably polyvinylpyrrolidone (each based on the total weight of the electrolyte). In this embodiment, the concentration of NaHCO₃ in said aqueous solution is less than 0.1 mol/L and the concentration of KCl in said aqueous solution is less than 0.2 mol/L.

Such an electrolyte composition is particularly suitable for measuring CO₂ according to the Stow-Severinghaus principle.

In another preferred embodiment, the electrolyte comprises less than 35% of said hygroscopic compound, preferably glycerol, and more than 60% of said hydrophilic evaporation-inhibiting compound, preferably polyethylene glycol, less than 10% of an aqueous solution of NaHCO₃ and KCl, and less than 2% of said surfactant, preferably polyvinylpyrrolidone (each based on the total weight of the electrolyte). In this embodiment, the concentration of NaHCO₃ in said aqueous solution is less than 0.1 mol/L and the concentration of KCl in said aqueous solution is less than 0.2 mol/L.

Such an electrolyte composition provides enough gas solubility for measuring oxygen (O₂) gas by optical method, when optical sensing element is covered by thin layer of the electrolyte.

The object is further achieved by an electrochemical sensor for sensing at least one target gas, in particular carbon dioxide and/or oxygen.

According to the present invention, the electrochemical sensor comprises a measurement chamber comprising an electrolyte, an indicator electrode and a reference electrode, which are in contact with the electrolyte. The measurement chamber can have a cylindrical shape with a volume between 0.01 to 4 mm³. The sensor may optionally further comprise an optical module covered with fluorescent indicator for reactive oxygen sensing. The electrochemical sensor further comprises a gas-permeable membrane through which a sample gas comprising the target gas can penetrate into the electrolyte. In other words, the gas-permeable membrane is a contact surface between the measurement chamber and the environment. In order to be able to attach the sensor to a living body the sensor also includes means for attaching the electrochemical sensor to the living body. Such means for attaching the electrochemical sensor to the living body include, e.g. fasteners configured for form-fit and/or frictional connection with a coupling receptacle which is glued, clamped and/or strapped to the living body's skin. In addition or alternatively, the electrochemical sensor itself can be glued, clamped and/or strapped to the living body's skin. The electrolyte in the electrochemical sensor disclosed herein comprises at least one hygroscopic compound comprising at least two moieties (HB) having hydrogen bonding capability and at least one hydrophilic evaporation-inhibiting compound comprising a carbon-to-HB ratio of ≥2 and a molecular weight MW of more than 100 g/mol. The evaporation-inhibiting compound comprises a lower viscosity and a lower surface tension than said hygroscopic compound. The hygroscopic compound of the electrolyte composition disclosed herein may additionally or alternatively have a proton diffusivity of more than 10⁻⁷ cm²/s at 25° C.

An electrochemical sensor as disclosed herein has the advantage that it is configured for multiple measurements, has a fast response time for the analyte to be determined, requires little maintenance and shows only a small drift, even after prolonged measurements, including changes from wet to dry gas exposure. The electrochemical sensor disclosed herein is thus particularly suitable for applications in which a large number of samples are to be measured with one sensor or in which a continuous control of the presence and/or the amount of an analyte is to be carried out over a longer period of time of e.g. 1 week or longer, especially 1 month or longer, such as but not limited to the transcutaneous measurement of blood gases.

Preferably, the at least one hygroscopic compound of the electrolyte used in the electrochemical sensor disclosed herein comprises a carbon-to-HB ratio of ≤2, more preferably ≤1.5, even more preferably ≤1.34, most preferably 1.0. Optionally, said hygroscopic compound may preferably have a proton diffusion coefficient of more than 10⁻⁶ cm²/s at 25° C.

Preferably, the at least one hydrophilic evaporation-inhibiting compound used in the electrochemical sensor disclosed herein comprises a molecular weight MW of more than 400 g/mol.

Preferably, the electrolyte used in the electrochemical sensor is an embodiment of the electrolyte disclosed herein.

The use of an electrolyte as disclosed herein in the electrochemical sensor disclosed herein has the advantages set out above for the electrolyte composition.

The indicator electrode and the reference electrode of the electrochemical sensor disclosed herein may be made of any material, which is suitable for the purposes of the potentiometric measurement. The reference electrode can be with or without liquid junction and is preferably formed from Ag/AgCl. Ag or Ag/AgCl is suitable as (quasi) reference electrode for sensors for the potentiometric determination of carbon dioxide by means of a pH electrode according to the Severinghaus principle.

The gas-permeable membrane allows the gaseous analyte to penetrate the electrochemical sensor, but is intended in particular to prevent the entry of ions and/or non-volatile components of the (aqueous) measuring medium in the electrolyte. As transcutaneous blood gas sensors locally heat the skin to typically 38 to 43° C. to induce vasodilation and maximize correlation between arterial pCO₂ and pCO₂ measured on skin, the gas-permeable membrane must also ensure heat transport to the tissue. The gas-permeable membrane can thus be made of any material that is suitable for such purposes. By way of example, the gas-permeable membrane comprises at least one polymeric material, wherein siloxanes, polytetrafluoroethylene and/or copolymers of tetrafluoroethylene, have proven to be particularly advantageous membrane materials for this purpose. The gas permeable membrane can be exchangeable or permanently set over the electrolyte.

The gas-permeable membranes used in an electrochemical sensor according to the present invention typically have a thickness of 5 μm to about 50 μm, preferably from about 10 μm to about 25 μm.

The thickness of the gas-permeable membrane represents a trade-off between the response time of the sensor and the robustness of the membrane itself, whereby the response time decreases with increasing membrane thickness.

For the determination of the target gas, the electrochemical sensor disclosed herein can be designed in any way that allows contact between the electrochemical sensor and the measuring medium. To this end, the electrochemical sensor comprises means for attaching the electrochemical sensor to a living body as will be explained in detail hereinafter based on exemplary embodiments.

In a further aspect of the present invention, the object is achieved by a method of sensing a partial pressure of a target gas in a sample gas. According to the invention, the method comprises the steps of introducing the sample gas to an electrochemical sensor as described herein and outputting an electrical signal from the electrochemical sensor representative of the target gas partial pressure.

Depending on the presence and/or the amount of the analyte, a measurable electrical signal is generated by the sensor. Preferably, this electrical signal, such as electrical current, voltage, resistance, etc., is evaluated or read out using suitable means. Preferably the electrochemical sensor is a potentiometric sensor, as in the case of a carbon dioxide sensor with a (Stow-)Severinghaus-type electrode. Oxygen gas is preferably measured optically through a photodiode, detecting the electrical current out of the decay of florescence from an excited pigment coated with electrolyte.

The object is further achieved by the use of an electrolyte as described herein for an electrochemical sensor, in particular as described herein, for detecting at least one target gas. Preferably, said target gas is selected from oxygen and/or carbon dioxide.

The following examples are intended to explain the present invention without restricting it. Unless otherwise indicated, all quantities are based on the total weight of the respective electrolyte formulations.

### Electrolyte Compositions for Sensing Only CO₂

The electrolyte compositions for sensing CO₂ through electrochemical sensing relies on potentiometric measurement.

| INCI | Example 1 | Example 2 |
|---|---|---|
| Glycerol | 50 | 10 |
| PEG-200 | 30 | 0 |
| PEG-150 | 0 | 80 |
| PVP; Povidone K25 | 4 | 0 |
| PVP; Povidone K90 | 0 | 0.2 |
| NaHCO₃ | 0.05 mol/L | 0.04 mol/L |
| KCl | 0.2 mol/L | 0.05 mol/L |
| Water | 15 | 9 |

Electrolyte Compositions for Sensing CO₂ and O₂

The electrolyte composition is suitable for sensing O₂ through an optical method. The same electrolyte is also suitable for sensing CO₂ and O₂ simultaneously via electrochemical and optical sensing respectively.

| INCI | Example 3 |
|---|---|
| Glycerol | 15 |
| PEG-100 | 62 |
| PEG-150 | 15 |
| PVP; Povidone K25 | 0.5 |
| NaHCO₃ | 0.05 mol/L |
| KCl | 0.05 mol/L |
| Water | 7 |

The invention is further explained in more detail by means of drawings, in which like reference numerals are used for like and corresponding parts of the accompanying drawings.

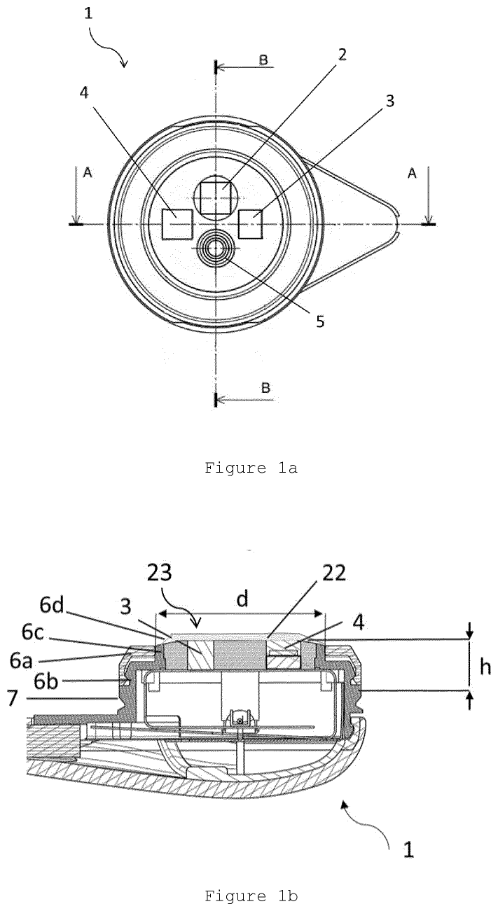

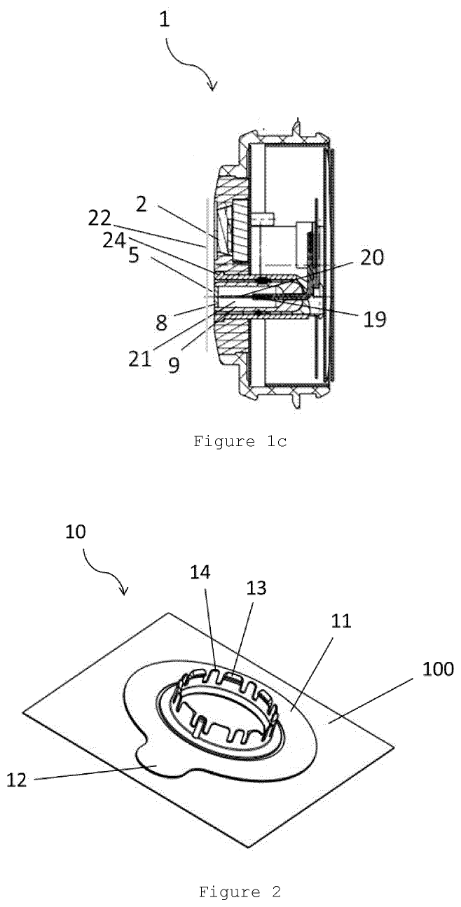

FIG. 1a: Top view of the measuring side of a sensor according to the invention configured for transcutaneous measurements;

FIG. 1b: Cross section of the sensor from FIG. 1a along the line A-A in FIG. 1a;

FIG. 1c: Cross section of the sensor from FIG. 1a along the line B-B in FIG. 1a (membrane ring with membrane (6a and 6d) not shown);

FIG. 2: Perspective view of a multi-site coupling receptacle for the sensor from FIG. 1a;

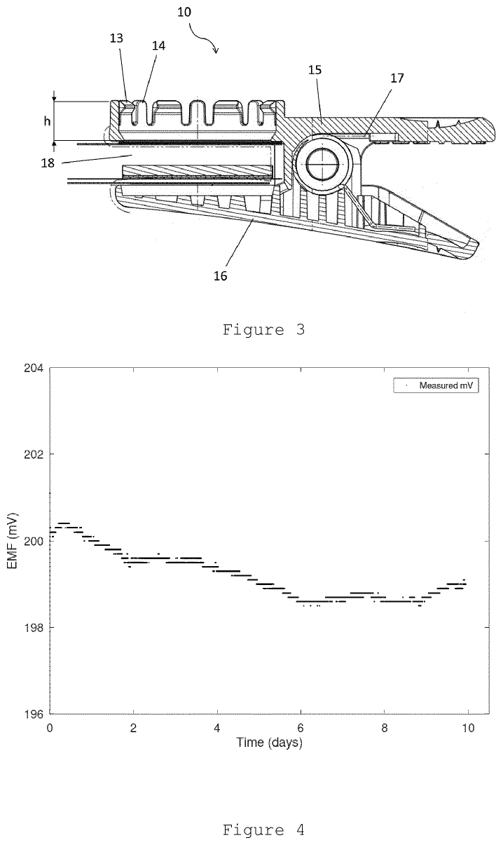

FIG. 3: Cross section of a mounting clip comprising a coupling receptacle for electrochemical sensor according to the invention;

FIG. 4: Plot showing potentiometric measurement results obtained with electrochemical sensors according to the invention for long time exposure to wet gas with a constant CO₂ partial pressure;

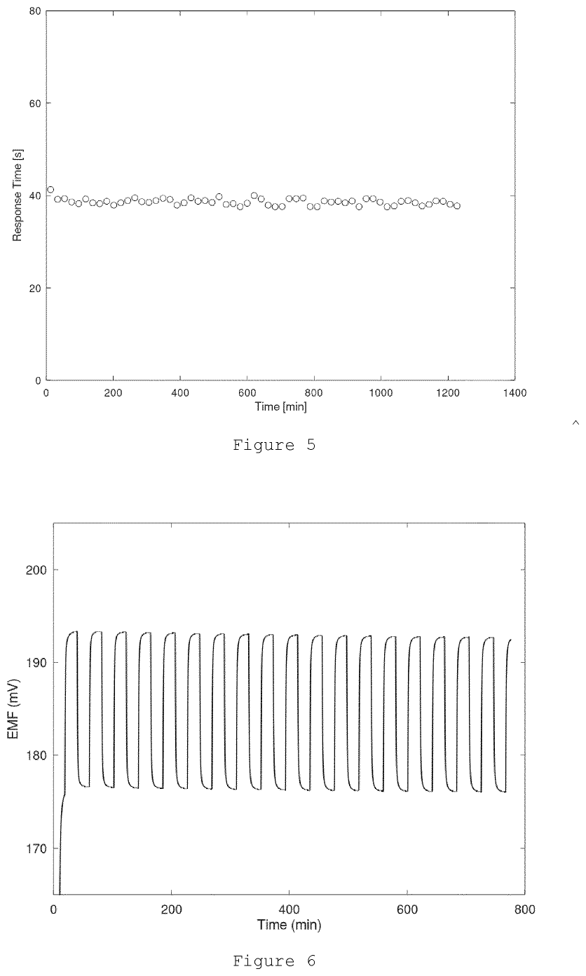

FIG. 5: Response time of a freshly applied electrolyte on an electrochemical sensor according to the invention for a 5 to 10% CO₂ concentration gas swing.

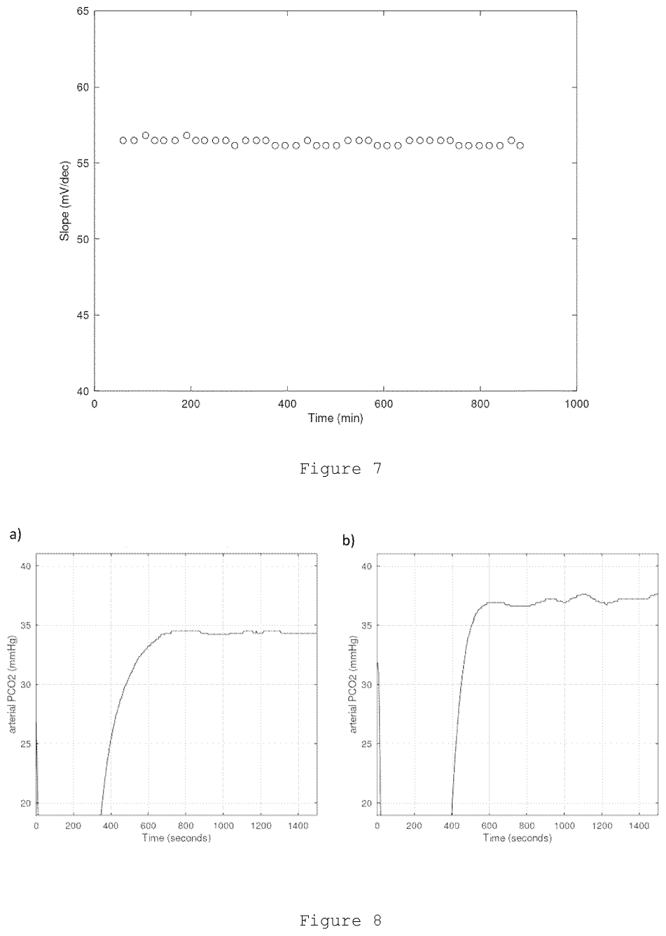

FIG. 6: Stability of the electrolyte: Response of an electrochemical sensor to a gas swing of 5 to 10% CO₂ concentration, 8 months after application of electrolyte on the electrochemical sensor according to the invention.

FIG. 7: Nernstian slope for the Severinghaus electrode, 8 months of application of electrolyte according to the invention.

FIG. 8: a) Estimated arterial blood gas based on a transcutaneous measurement on a healthy adult with the sensor according to the invention, 11 months after application of the electrolyte onto the sensor;
b) Estimated arterial blood gas based on a transcutaneous measurement on a healthy adult with the sensor according to the invention and freshly applied electrolyte according to the invention.

FIG. 1a shows a measuring side of an electrochemical sensor 1 for the transcutaneous tc measurement of blood gases (no membrane ring mounted). In the embodiment of the sensor 1 shown here, only the carbon dioxide partial pressure PCO₂ is measured by a tcPCO₂ electrochemical module (indicator electrode) 5, whereas the oxygen partial pressure PO₂ is determined by a tcPO₂ optical module 2, a pulse oximetry light-emitting diode 3 and a pulse oximetry photodiode 4 (optodes). In the embodiment depicted in FIG. 1a, the electrolyte covers the whole surface of modules 2, 3, 4 and 5, as will be better understood from FIG. 1b as 22. The sensor housing of the electrochemical sensor 1 is configured as a coupling element, which can be brought into engagement with a coupling receptacle 10, i.e. means for attaching the electrochemical sensor 1 to the living body, as will be described in more detail in FIGS. 1b and 1c.

In the context of the present specification, the term “multi-site” refers to the application site of the coupling receptacle on different locations of the living body. By way of example, for CO₂ measurement the coupling receptacle is attached to the thorax, abdominal side, thigh, subclavian, forehead, ear lobe and cheek of the living body. FIG. 1b shows a cross section of the sensor 1 from FIG. 1a along the line A-A in FIG. 1a, which runs through optodes 3 and 4. The membrane ring 6a, the position of which is given for orientation only, is attachable to groove 6b. Means for attaching the electrochemical sensor 1 to a living body include an engagement groove 6b and retention element 7. Both 6b and 7 run along the radial outer surface of the sensor 1.

Ideally, the electrolyte 22 covers the full surface shown with the measure d and is itself covered by the membrane. Alternatively, the electrolyte 22 can cover electrochemical module (ref. sign 5 in FIG. 1a) alone or in combination with the other modules.

The membrane ring 6a is lockable into groove 6b, implements a pressure seal on flank 6c and stretches over section d with a diameter of ca. 10 mm and an area of less than 100 mm². The membrane can be temporary or permanently fixed. The membrane might consist of a single layer or multi-layers. The layer on top of module 5 should be gas permeable and would be out of organic CO₂ permeable material, porous metallic compounds or any combination of both in a composite form. The layer outside of the module 5 would be out of materials from class of polymeric, metallic, non-metallic, organic, inorganic or any combination of these. For sensing O₂, the layer on top of module 2 should be oxygen permeable.

To engage the sensor 1 with the coupling receptacle 10, the sensor 1 is inserted into the coupling receptacle 10 such that the engagement elements 13 of the coupling receptacle 10 engage with the engagement groove 6b of the coupling element.

When the coupling element and the coupling receptacle 10 are engaged, the actual measurement modules should lie as flat as possible on the skin of the living body and are slightly pressed onto it. As can be seen from the cross sections shown in FIGS. 1b and 1c, the sensor 1 features a slightly convex bottom surface facing the living body's skin to ensure skin contact. In the present example, the sensor 1 is inserted in the coupling receptacle 10 approximately to the height h. The diameter d of the engagement element is chosen such that the sensor 1 can be accommodated in the coupling receptacle 10 with an essentially exact fit, wherein the sensor 1 can still be rotated in the coupling receptacle 10 when connected. In the present embodiment, the diameter d shown in FIG. 1b is approx. 10 mm and the insertion height h is approx. 4 mm.

FIG. 1c shows a cross section of the sensor 1 from FIG. 1a along the line B-B in FIG. 1a, which runs through tcPO₂ optical module 2 and tcPCO₂ electrochemical module 5. The tcPCO₂ electrochemical module 5 comprises a cationic exchange glass membrane 8 with ca. 3 mm in diameter, enabling selective proton transport and glass wall 9 containing an inner standard electrolyte 21, with the inner working electrode (Ag/AgCl) 20 attached to platinum electrode 19. The inner standard electrolyte 21 may be chosen by the skilled person and does not necessarily have to be an electrolyte as described herein. The sensor 1 further comprises a reference electrode 24, which in the embodiment shown in the figures is designed as an Ag/AgCl electrode. The thickness of the electrolyte layer 22 is about 5 μm to 10 μm when freshly applied over section d shown in FIG. 1b, corresponding to a volume of approximately 0.4 mm³ to 0.8 mm³. The exposed surface to volume ratio there is in the range of 100 mm⁻¹ to 200 mm⁻¹, preferably 130 mm⁻¹ to 170 mm⁻¹, most preferably 150 mm⁻¹. Also shown are the circumferential membrane engagement groove 6b and the circumferential retention element 7 described in FIG. 1b.

FIG. 2 shows a perspective view of a multi-site coupling receptacle 10 for the sensor 1 from FIG. 1a. In this embodiment, the coupling receptacle 10 comprises an adhesive pad 11 that can be applied to a living body's skin 100. The coupling receptacle 10 further comprises a number of engagement elements 13, preferably eight engagement elements 13, which can be inserted into engagement grooves 6b of a coupling element of a sensor 1. The engagement grooves 6b are preferably arranged on a region of the coupling element, which tapers towards the direction of the coupling receptacle 10. The sensor 1 is further supported by retaining elements 14, which limit and support the mobility of the sensor 1 in addition to the engagement elements 13 in lateral direction. In the present embodiment, both the engagement elements 13 and the retaining elements 14 are arranged in a circle. The inner diameter of the circle defined by these elements 13, 14 is approx. 14 mm and the height of these elements 13, 14 is approx. 4 mm.

In order to measure on the earlobe or skin flap of a living body, FIG. 3 shows a cross section of a mounting clip comprisesing a coupling receptacle 10, wherein the coupling receptacle 10 features engagement elements 13 and retaining elements 14, which are both arranged in a circle. In this embodiment, the height h of the engagement elements 13 and the retaining elements 14 is approx. 4 mm. The inner diameter of the circle defined by these elements 13, 14 is approx. 14 mm. The mounting clip is configured for attachment to the earlobe or skin flap of a living body and to this end comprises a clamping opening 18 defined by the upper part 15 and the lower part 16 of the coupling receptacle 10, into which the e.g. the earlobe or a skin flap of a living body can be inserted and clamped by means of a torsion spring 17 or another friction-locked fastener.

A coupling receptacle of this type enables a secure and simple connection of a sensor to a living body's earlobe in order to measure blood gases securely and reliably. The work required for a user is simplified and reduced, and process reliability is increased.

FIG. 4 shows the evolution of potentiometric measurements of humidified 10% CO₂ gas performed with a sensor and freshly applied electrolyte according to the invention for gas exposure over the course of several days. As apparent from the recorded voltages, the drift of the CO₂ measurements remains below 2% over a period of 7 days. Notably, no drift-correcting algorithm was applied. Both the long-term stability of the measurement and the short-term drift behaviour are excellent for clinical application.

FIG. 5 shows the response time of a CO₂ sensor according to the invention for dry gas exchange of 5% to 10% CO₂ composition with a freshly applied electrolyte of the invention. The response time (T₉₀) is defined as the time required by the sensor to show a shift from 10% to 90% of the final stable value of CO₂ partial pressure. The response times matches the in-vitro performance of the available products in the market for monitoring physiological changes in clinical application.

FIG. 6 shows the evolution of potentiometric measurement when the dry gas CO₂ concentration shifts from 5 to 10% at ambient temperature. The data corresponds to an electrolyte residing on a sensor as per the embodiments of this invention for over 8 months. During this time the sensor was performing transcutaneous gas measurements according to the embodiments of this invention. Furthermore, the drift in the measurement resides below 2%, indicating that aging barely affected the sensitivity and low-drift behaviour of the electrolyte as per this invention, despite of multiple measurements at clinical condition without any maintenance.

FIG. 7 shows the Nernstian slope of a sensor working based on a Severinghaus principle. The slope is measured by shifting CO₂ concentration from 5 to 10% CO₂ at ambient condition for 800 min after electrolyte resided on sensor for 8 months. One can identify significant changes in the electrolyte composition upon aging by evaluating the difference of this slope from ideal theoretical Nernstian value of 62.5 mV/decade at 42° C. As it is shown in FIG. 7, the slope value remained in the 57 mV range, reflecting stability of electrolyte composition over 8 months of measurement.

FIG. 8 demonstrates estimated arterial carbon dioxide blood gas from a transcutaneous measurement on a healthy adult carried out by an electrolyte and sensor of a preferred embodiment of this invention, where (a) electrolyte resided over 11 months on the sensor and (b) freshly applied electrolyte. The initial increase in the measured value corresponds to the stabilization of the sensor to equilibrate with skin temperature and CO₂ concentration. The data is derived from raw physical measurements and not corrected by any algorithmic correction. In FIG. 8a the electrolyte had more than 11 months age from application time on the sensor without any maintenance in between. During this time, the sensor either was under periodical dry calibration gas or was set on healthy adults for multiple transcutaneous measurements. The measured value lies within the expected values of 30 to 40 mmHg for a healthy person, without drift algorithmic corrections. FIG. 8b demonstrates the results of same type of measurement as of 8a, but with a freshly applied electrolyte. These two measurements do not reflect significant change within acceptable performance of physiological relevance within period of 11 months aging time of the electrolyte. These facts indicate that the electrolyte according to this invention remained clinically functional without any required maintenance, reflecting the electrolyte stability for almost a year.

Overall, the observed performance of the electrolyte as per preferred embodiment of this invention fulfil the required properties for long term stable measurement without a need for maintenance, assuming other parts of the sensor stay stable as well.

1. An electrolyte composition for use in an electrochemical sensor, said electrolyte composition comprising:
at least one hygroscopic compound comprising at least two moieties (HB) having hydrogen bonding capability and preferably a carbon-to-HB ratio of ≤2, more preferably ≤1.5, even more preferably ≤1.34, most preferably 1.0; and
at least one hydrophilic evaporation-inhibiting compound comprising a carbon-to-HB ratio of ≥2 and a molecular weight (MW) of more than 100 g/mol, preferably more than 400 g/mol,
wherein said evaporation-inhibiting compound comprises a lower viscosity and a lower surface tension than said hygroscopic compound.

2. The electrolyte according to claim 1, wherein said moieties having hydrogen bonding capability are at least one of hydroxyl, carboxyl, amine, imine, and/or amide groups,
wherein said hygroscopic compound is preferably selected from the group consisting of polyhydric alcohols, glycerol, triglycerol, polyglycerol, diethylene glycol, triethylene glycol, triethanolamine, diethanolamine and/or propylene glycol, more preferably polyhydric alcohols, glycerol, triglycerol, diethylene glycol, triethylene glycol and/or diethanolamine, most preferably glycerol and/or diethylene glycol; and/or
wherein the hygroscopic compound is preferably present in the electrolyte in an amount of less than 90% based on the total weight of the electrolyte.

3. The electrolyte according to claim 1, wherein the evaporation-inhibiting compound is selected from the group consisting of short chain oligomers of polyethylene glycol, polyglycerol, polyoxazolines, polyhydroxy-functional acrylates, in particular poly(2-hydroxyethyl methacrylate), polyethylene oxide, hydrophilic polycarbonates and/or co-, graft- and block copolymers thereof, and wherein the evaporation-inhibiting compound is preferably present in the electrolyte in an amount of at least 10% based on the total weight of the electrolyte.

4. The electrolyte according to claim 1, wherein the electrolyte further comprises a surfactant, preferably a non-ionic and non-metallic surfactant.

5. The electrolyte according to claim 4, wherein said surfactant is selected from the group consisting of polyvinylpyrrolidone (PVP), polyvinyl alcohol (PVA), polysaccharides, and co-graft- and/or block copolymers thereof, preferably said surfactant is selected from the group consisting of polyvinylpyrrolidone (PVP), polyvinyl alcohol (PVA), polysaccharides and co-, graft- and/or block copolymers thereof,
wherein the surfactant is preferably present in the electrolyte in an amount of less than 5%, more preferably in an amount of less than 2%, based on the total weight of the electrolyte.

6. The electrolyte according to claim 1, wherein the electrolyte further comprises a thickening agent, preferably a non-ionic and non-metallic thickening agent.

7. The electrolyte according to claim 6, wherein said thickening agent is selected from the group consisting of polyvinylpyrrolidone (PVP), polyethylene derivatives, polyvinyl alcohols, poly-(2-vinylpyridines), polyethylene oxides, polyethylene glycol monomethyl ethers, polyvinyl methyl ethers, polysaccharides, polycarbonates, and combinations thereof, preferably said thickening agent being selected from the group consisting of polyvinylpyrrolidone (PVP), polyethylene derivatives, polysaccharides, polycarbonates, and combinations thereof,
wherein said thickening agent is preferably present in the electrolyte in an amount of less than 5%, more preferably in an amount of less than 2%, based on the total weight of the electrolyte.

8. The electrolyte according to claim 1, wherein the electrolyte further comprises an aqueous solution of at least one inorganic bicarbonate-based pH buffer, in particular with a buffer range between pH 7 and 9, and,
optionally, at least one chloride salt, wherein the concentration of said chloride salt in the aqueous buffer solution is less than 3 mol/L, preferably less than 1 mol/L.

9. The electrolyte according to claim 8, wherein said bicarbonate-based pH buffer is selected from the group consisting of sodium bicarbonate buffer and/or potassium bicarbonate buffer, and/or wherein said chloride salt is selected from the group consisting of LiCl, NaCl, and/or KCl.

10. The electrolyte according to claim 1, wherein said evaporation-inhibiting compound has an oxygen solubility of more than 0.01 mg/L and less than 10 mg/L at 25° C. and 1 bar and/or wherein all compounds except water have a vapor pressure smaller than 0.1 mmHg at 25° C.

11. The electrolyte according to claim 10, in which the electrolyte comprises:
less than 90% of said hygroscopic compound, preferably glycerol;
more than 10% of said hydrophilic evaporation-inhibiting compound, preferably polyethylene glycol;
less than 10% of an aqueous solution of NaHCO₃ and KCl, wherein the concentration of NaHCO₃ in said aqueous solution is less than 0.1 mol/L and the concentration of KCl in said aqueous solution is less than 0.2 mol/L; and
less than 5% of said surfactant, preferably polyvinyl-pyrrolidone;
each based on the total weight of the electrolyte.

12. The electrolyte according to claim 4, wherein the electrolyte comprises:
less than 35% of said hygroscopic compound, preferably glycerol;
more than 60% of said hydrophilic evaporation-inhibiting compound, preferably polyethylene glycol; and
less than 2% of said surfactant, preferably polyvinyl-pyrrolidone;
each based on the total weight of the electrolyte.

13. A sensor for sensing at least one target gas, in particular for sensing carbon dioxide electrochemically and optionally oxygen optically, the electrochemical sensor comprising:
a measurement chamber comprising an electrolyte, in particular an electrolyte according to claim 1;
an indicator electrode and a reference electrode in contact with the electrolyte;
an optional optical module covered with fluorescent indicator for reactive oxygen sensing;
a gas-permeable membrane through which a sample gas comprising the target gas can penetrate into the electrolyte; and
means for attaching the electrochemical sensor to a living body, in particular the sensor is configured for engagement to a coupling receptacle, preferably a fastening clip or an adhesive holder, attached to a living body;
wherein said electrolyte comprises:
at least one hygroscopic compound comprising at least two moieties (HB) having hydrogen bonding capability and preferably a carbon-to-HB ratio of ≤2, more preferably ≤1.5, even more preferably ≤1.34, most preferably 1.0;
at least one hydrophilic evaporation-inhibiting compound comprising a carbon-to-HB ratio of ≥2 and a molecular weight MW of more than 100 g/mol, preferably more than 400 g/mol; and
wherein
said evaporation-inhibiting compound comprises a lower viscosity and a lower surface tension than said hygroscopic compound.

14. A method of sensing a partial pressure of a target gas in a sample gas, the method comprising the steps of:
introducing the sample gas to an electrochemical sensor according to claim 13;
outputting an electrical signal from the electrochemical sensor representative of the target gas partial pressure.

15. Use of an electrolyte according to claim 1 for an electrochemical sensor for detecting at least one target gas, in particular oxygen and/or carbon dioxide.
